(19) United States
(12) Patent Application Publication
(10) Pub. No.: US 2004/0158232 A1
(43) Pub. Date: Aug. 12, 2004

(54) Implantable artificial pancreas

(76) Inventors: Laurence MacDonald Schetky, Camden, ME (US); Francis Moussy, Tampa, FL (US); Peter Jardine, Thousand Oaks, CA (US)

Correspondence Address: CANTOR COLBURN, LLP, 55 GRIFFIN ROAD SOUTH, BLOOMFIELD, CT, 06002

(21) Appl. No.: 10/638,193

(22) Filed: Aug. 7, 2003

Related U.S. Application Data
(60) Provisional application No. 60/401,674, filed on Aug. 7, 2002.

(51) Int. Cl. A61K 9/22

(52) U.S. Cl. 604/890.1

(57) ABSTRACT

An artificial pancreas comprises a first reservoir for retaining insulin; at least one second reservoir for retaining a therapeutic agent; at least one pump in fluid communication with the first reservoir and the at least one second reservoir; and a glucose monitor in electrical communication with the pump.

### CROSS REFERENCE TO RELATED APPLICATIONS

This application claims the benefit of U.S. Provisional Application Serial No. 60/401,674 filed on Aug. 7, 2002, the entire contents of which are hereby incorporated by reference.

### BACKGROUND

This disclosure relates to an implantable artificial pancreas. In particular, this disclosure relates to a closed loop insulin delivery system that is implantable and functions as an artificial pancreas.

The control of Type I Diabetes Mellitus is generally effected by the periodic injection of insulin to maintain blood glucose levels as close to normal as possible. The blood glucose level is monitored by means of a device that directly measures glucose from a blood sample. Insulin is injected in the appropriate quantities and at the appropriate intervals to correct imbalances in the blood glucose level. Careful control of blood glucose levels is mandatory for preventing the onset of complications such as retinopathy, nephropathy and neuropathy. Unfortunately in many cases, patients neglect to perform regular glucose monitoring and therefore suffer episodes of hyperglycemia or hypoglycemia, which may, in turn, lead to the complications listed above or death.

Blood-glucose levels generally vary with activity or food intake and insulin is therefore administered by sub-cutaneous hypodermic injection to minimize variations in the blood glucose levels that generally occur with activity or food intake. Small externally worn pumps are also available to deliver insulin percutaneously, thereby replacing the tedious use of a hypodermic injection, but constant glucose monitoring is still an important component of control. Attempts to develop a closed loop system for the control of glucose levels have led to the development of ever more sophisticated insulin pump systems, but an accurate long lived implanted blood glucose level monitor that would provide the required signal for a closed loop insulin pump control is not yet available. At the present time the subcutaneous implanted monitors which have been investigated function for days or even weeks but ultimately fail due to tissue inflammation reactions at the monitor site.

### SUMMARY

An artificial pancreas comprising a first reservoir for retaining insulin; at least one second reservoir for retaining a therapeutic agent; at least one pump in fluid communication with the first reservoir and the at least one second reservoir; and a glucose monitor in electrical communication with the pump.

### BRIEF DESCRIPTION OF FIGURES

FIG. 1 represents a schematic representation of one exemplary embodiment of the artificial pancreas;

FIGS. 2(a) and 2(b) shows one exemplary embodiment of the pump 20A, which comprises a thin film of a shape memory alloy 200 deposited upon a substrate 202 having a circular cavity 204;

FIG. 3 is a schematic representation depicting the hot deformation of the film 200 to impart to the film a dome shape, which the film will assume when heated.

FIG. 4 is a schematic representation depicting one exemplary mode of working of the pump;

FIG. 5 is a schematic representation of one embodiment of a duplex pump;

FIG. 6 is a schematic representation of one exemplary embodiment of the artificial pancreas containing a duplex pump;

FIG. 7 is a schematic representation of one embodiment of a glucose sensor;

FIG. 8 is a schematic representation of the electronic control system, which forms the interface between the glucose monitor and the plump;

FIG. 9 is a graphical representation of the amount of dexamethasone released into the PBS (phosphate buffered saline solution) as a percentage of the total amount of dexamethasone encapsulated into the microsphere system as a function of the elapsed time from the beginning of the release studies; and

FIG. 10 is a micrograph showing the effect of empty PLGA microspheres and microspheres having dexamethasone on an inflammatory response to thread sutures.

### DETAILED DESCRIPTION OF PREFERRED EMBODIMENTS

Disclosed herein is an artificial pancreas comprising a duplex pump which can dispense insulin for maintaining blood glucose levels at a desired value and additionally can dispense a therapeutic agent to the site of implantation of a glucose monitor to reduce tissue inflammatory response. The artificial pancreas further comprises an implantable glucose monitor that can advantageously function for an extended period of time when implanted subcutaneously in a living being. The artificial pancreas also comprises suitable electronics that in conjunction with the pump and the glucose monitor form a closed loop system. The artificial pancreas can advantageously be implanted into the body of a living being and can function without maintenance or removal from the body for a time period greater than or equal to about 1 month, preferably greater than or equal to about 6 months, and more preferably greater than or equal to about 12 months.

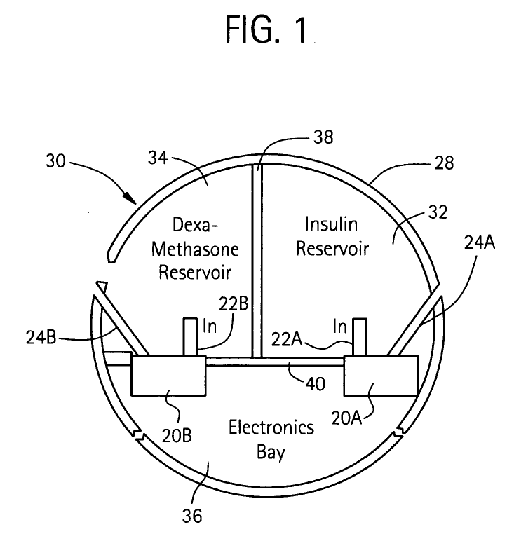

As stated above, the artificial pancreas comprises at least one pump, a glucose monitor and the associated electronics, which form a closed loop system that can maintain blood glucose levels at a desired value and additionally reduce the tissue inflammatory response. As shown in the schematic in FIG. 1, in one exemplary embodiment, the artificial pancreas 30 comprises a housing 28 which encapsulates a first reservoir 32 that can retain insulin and is in fluid communication with a first pump 20A having an inlet port 22A and an exit port 24A, at least one second reservoir 34 that can retain a therapeutic agent and is in fluid communication with a second pump 20B having an inlet port 22B and an exit port 24B, and an electronics bay 36 that contains the control electronics that provides the interface between the glucose monitor and the insulin pump. The first reservoir 32 and the at least one second reservoir 34 are separated from each other by a partition 38 and are not in fluid communication with one another. The first reservoir 32 and the second reservoir 34 are also respectively separated from the electronics bay 36 by another partition 40. The control electronics permit the pump to respond to the demand for insulin from the glucose monitor. The pumps 20A and 20B are also in fluid communication with a first check valve (not shown) which facilitates the flow of fluid from the reservoirs 32, 34 to the pumping cavity and a second check valve (not shown) which facilitates the flow of the pumped fluid from the pump cavity to the delivery tube. The first check valve and second check valve used for controlling the flow of fluid into and out of the pump are either ball check valves or disc type check valves. The film is in electrical communication with a battery, which provides the electrical current for resistive heating of the film.

The housing 28, partition 38 and partition 40 preferably comprises materials that are biocompatible and through which a hypodermic syringe can be introduced for purposes of replenishing the reservoirs with glucose and the therapeutic agent if desired. Examples of suitable therapeutic agents include anti-inflammatory agents such as dexamethasone, prednisolone, corticosterone, budesonide, estrogen, sulfasalazine, mesalamine, or the like. The preferred anti-inflammatory agent is dexamethasone.

The therapeutic agent can be genetic, non-genetic or may comprise cells or cellular matter. Examples of non-genetic therapeutic agents are antithrombogenic agents such as heparin and its derivatives, urokinase, and dextropheylalanine proline arginine chloromethylketone (Ppack); anti-proliferative agents such as enoxaprin, andiopeptin, or monoclonal antibodies capable of blocking smooth muscle cell proliferation, hirudin, and acetylsalicylic acid; antineoplastic/antiproliferative/anti-miotic agents such as paclitaxel, 5-fluorouracil, cisplatin, vinblastine, vincristine, epothilones, endostatin, angiostatine and thymidine kinase inhibitors; anesthetic agents such as lidocaine, bupivacaine, and ropivacaine; anti-coagulants such as D-Phe-Pro-Arg chloromethyl keton, an RGD peptide-containing compound, heparin, antithrombin compounds, platelet receptor antagonists, anti-thrombin anticodies, anti-platelet receptor antibodies, aspirin, prostaglandin inhibitors, platelet inhibitors and tick antiplatelet peptides; vascular cell growth promoters such as growth factor inhibitors, growth factor receptor antagonists, transcriptional activators, and translational promoters; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; and agents which interfere with endogenous vascoactive mechanisms.

In one embodiment, the housing 28 comprises at least one port (not shown) through which additional glucose and anti-inflammatory agent may be added to the first reservoir 32 and the second reservoir 34 respectively for purposes of replenishing the supply. In another embodiment, the housing 28 comprises polymeric resinous materials that are self-curing, wherein the housing upon being impaled by a hypodermic syringe for the purpose of replenishing the glucose or anti-inflammatory agent into the respective reservoirs may undergo self-curing to eliminate the cavity in the housing created by the introduction of the syringe. In one embodiment, the partition 38 and the partition 40 generally comprise the same metallic or non metallic biocompatible materials as the housing 28 if desired. In another embodiment, the partition 38 and the partition 40 generally comprise different metallic or non metallic biocompatible materials as the housing 28 if desired. In yet another embodiment, the partition 38 and the partition 40 may comprise different materials from one another. In yet another embodiment, the partition 40 may comprise a first biocompatible material for the first reservoir 32 and a second biocompatible material for the second reservoir 34. In yet another embodiment the design can encompass more than one second reservoir for retaining multiple therapeutic agents. In such a case, the additional reservoirs may be designated as a third reservoir, fourth reservoir and so on, depending upon the number of reservoir. All such reservoirs may be in fluid contact with the pump and can be isolated from one other if desired. If desired, some or all of these additional reservoirs may be in fluid communication with one another through check valves and other associated fluid handling devices such a pumps, gages, valves, nozzles, orifices, and the like.

Suitable examples of such metallic biocompatible materials that may be used for the housing 28, partition 38 and partition 40 are titanium or titanium alloys such as nitinol, stainless steel, tantalum, and cobalt alloys including cobalt-chromium nickel alloys. Suitable nonmetallic biocompatible materials are polymeric resins such as polyamides, polytetrafluoroethylene, silicone polymers such as polydimethylsiloxane, polyolefins such as polyethylene and/or polypropylene, nonabsorbable polyesters such polyethylene terephthalate and/or polybutylene terephthalate and bioabsorbable aliphatic polyesters such as homopolymers and copolymers of lactic acid, glycolic acid, lactide, glycolide, para-dioxanone, trimethylene carbonate, E-caprolactone, or the like, or biocompatible combinations comprising at least one of the foregoing non-metallic biocompatible materials.

The housing preferably has a thickness of about 0.1 millimeter (mm) to about 2 millimeters in thickness. Within this range, it is desirable to have a thickness of greater than or equal to about 0.4 mm, preferably greater than or equal to about 0.5 mm. Also desirable within this range, is a thickness of less than or equal to about 1.9, preferably less than or equal to about 1.8, and more preferably less than or equal to about 1.5 mm in thickness.

The first reservoir 32 generally carries insulin and has a volume of about 5 to about 50 milliliters (ml). Within this range, the first reservoir may have a volume of greater than or equal to about 8, preferably greater than or equal to about 10 and more preferably greater than or equal to about 12 milliliters. Also desirable within this range is a volume of less than or equal to about 45, preferably less than or equal to about 40 and more preferably less than or equal to about 30 milliliters.

The second reservoir 34 generally carries the anti-inflammatory agent and has a volume of about 25 ml to about 40 ml. Within this range, the first reservoir may have a volume of greater than or equal to about 27, preferably greater than or equal to about 28, and more preferably greater than or equal to about 29 ml. Also desirable within this range is a volume of less than or equal to about 39, preferably less than or equal to about 38 and more preferably less than or equal to about 35 milliliters.

The first and second reservoirs 32 and 34 for insulin and the anti-inflammatory drug are refilled at intervals using a transcutaneous hypodermic injection. Since insulin analogs now have concentrations of about 40 to about 500 units of insulin per milliliter (ml), there is some flexibility in the choice of the reservoir volume and therefore the interval between refills. On exemplary embodiment of a device for permitting the use of transcutaneous hypodermic injection for purposes of replenishing the reservoirs 32 and 34 are described in U.S. Pat. Nos. 4,573,994 and 5,514,103 both of which are hereby incorporated by reference.

The first pump 20A is a positive displacement diaphragm pump that can deliver a precise desirable quantity of insulin at each stroke. It is generally desirable for the first pump 20A to operate at a frequency to maintain a uniform value of blood glucose levels within the body despite physical activity or food intake. It is generally desirable for the pump delivery speed to be limited only by the response time of the glucose monitor. This permits the pump to respond rapidly to a given signal from the glucose monitor. In one embodiment, it is desirable for the first pump 20A to operate at a frequency of greater than or equal to about 0.2, preferably greater than or equal to about 0.5 and more preferably greater than or equal to about 1 hertz (Hz). This feature makes it possible to accurately follow glucose changes and avoid deviations in the blood glucose levels of greater than or equal to about 5.5 millimoles/liter when glucose levels rise and fall as a result of meals or exercise.

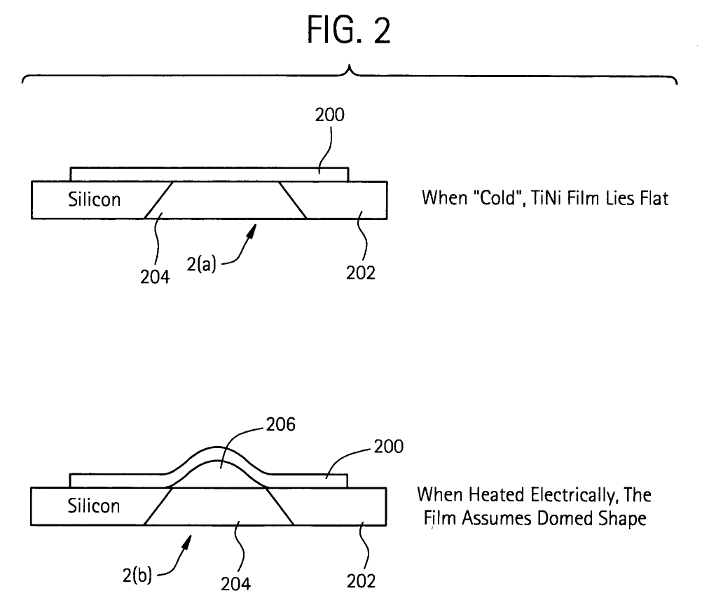

The pumps 20A and 20B generally deliver fluids at pressures of greater than or equal to about 25, preferably greater than or equal to about 50, and more preferably greater than or equal to about 75 pounds per square inch (psi). The capability of the first pump 20A to operate at a pressure of greater than or equal to about 75 psi minimizes catheter blockage due to precipitated insulin. The pump comprises a thin film of a shape memory alloy 200 which when cold lies flat and when heated assumes a dome shape and gives rise to a pumping action as is depicted in FIGS. 2(a) and 2 (b). FIG. 2(a) shows one exemplary embodiment of the pump 20A or 20B, which comprises a thin film of a shape memory alloy 200 deposited upon a substrate 202 having a circular cavity 204. Upon the application of heat to the shape memory alloy 200, it expands to form a dome as shown in FIG. 2(b), which creates an additional volume 206 that can be displaced when the film 200 is cooled and therefore returns to its original size and shape. The film 200 is generally heated and cooled by the sequential application and removal of a suitable pulse of an electrical current to the film that causes it to respectively rise and fall thereby giving rise to a pumping action.

Shape memory alloys generally undergo a martensitic transformation when cooled from some elevated temperature; the temperature difference separating the elevated temperature or parent phase from the low temperature martensitic phase varies with the alloy composition. When a shape memory alloy in the martensitic condition is deformed it will recover its original shape when heated to the temperature at which it transforms to the parent phase. If the specimen is again cooled it will not return to the previously deformed shape unless it is subjected to an externally applied force. In a typical shape memory actuator a shape memory spring is opposed by a conventional alloy spring, the so-called bias. When heated, the shape memory spring overcomes the biasing force and develops a net output force. When the shape memory spring cools, the bias spring can now force the shape memory spring to return to its original position because the martensite phase has a much lower modulus of elasticity that the parent phase. In the case of the film 200, which is to change from a flat to a dome shape to develop the pumping action, some form of biasing force will be required.

In a nickel titanium film possessing shape memory properties, control of the sputtering process can promote the composition of the deposited film to be varied from equiatomic to nickel rich. This composition gradient will exhibit shape memory while the nickel rich portion of the film will act as a restraining force or bias. When such a nickel titanium shape memory alloy film is deformed at high temperature, a predetermined shape such a dome is imprinted in the film. When the film cools the biasing layer of nickel forces the film into the flat position, but when the film is heated it returns to the dome shape imprinted by the hot deformation process.

The film 200 may be manufactured from a variety of shape memory alloys. The alloys used for the film 200 are preferably shape memory alloys having a reverse martensitic transformation start temperature (Aₛ) of greater than or equal to about 10° C. It is generally desirable for the film to have an Aₛ of greater than or equal to about 12° C., preferably greater than or equal to about 15° C., preferably greater than or equal to about 20° C., and more preferably greater than or equal to about 23° C. In another embodiment, the shape memory alloys used in film have an austenite transformation finish temperature (A_{f}) temperature of about 25° C. to about 40° C. Within this range, it is generally desirable to have an A_{f} temperature of greater than or equal to about 28° C., preferably greater than or equal to about 30° C. Also desirable within this range is an A_{f} temperature of less than or equal to about 38° C., preferably less than or equal to about 36° C.

Shape memory alloys that may be used in the films are generally nickel based titanium alloys. Suitable examples of nickel based titanium alloys are nickel-titanium-niobium, nickel-titanium-copper, nickel-titanium-iron, nickel-titanium-hafnium, nickel titanium zirconium nickel-titanium-palladium, nickel-titanium-gold, nickel-titanium-platinum alloys or the like, or combinations comprising at least one of the foregoing nickel based titanium alloys. Preferred alloys are nickel-titanium alloys, titanium-nickel-niobium and titanium-nickel-copper alloys.

Nickel-titanium alloys that may be used in the films generally comprise nickel in an amount of about 54.5 weight percent (wt %) to about 57.0 wt % based on the total composition of the alloy. Within this range it is generally desirable to use an amount of nickel greater than or equal to about 54.8, preferably greater than or equal to about 55, and more preferably greater than or equal to about 55.1 wt % based on the total composition of the alloy. Also desirable within this range is an amount of nickel of less than or equal to about 56.9, preferably less than or equal to about 56.7, and more preferably less than or equal to about 56.5 wt %, based on the total composition of the alloy.

An exemplary composition of a nickel-titanium alloy having an As greater than or equal to about 10° C. is one which comprises about 55.5 wt % nickel (hereinafter Ti-55.5 wt %-Ni alloy) based on the total composition of the alloy. The Ti-55.5 wt %-Ni alloy has an Aₛ temperature in the fully annealed state of about 30° C. After cold fabrication and shape-setting heat treatment, the Ti-55.5 wt %-Ni alloy has an Aₛ of about 10 to about 15° C. and an austenite transformation finish temperature (A_{f}) of about 30 to about 35° C.

Another exemplary composition of a nickel-titanium alloy having an Aₛ greater than or equal to about 0° C. is one which comprises about 55.8 wt % nickel (hereinafter Ti-55.8 wt %-Ni alloy) based on the total composition of the alloy. The Ti-55.8 wt %-Ni alloy generally has an Aₛ of 0° C. in its as-fabricated state, and an A_{f} of about 15 to about 20° C. However, upon subjecting the Ti-55.8 wt %-Ni alloy to aging through annealing, the Aₛ and A_{f} are both increased.

Nickel-titanium-niobium (NiTiNb) alloys that may be used in the film generally comprise nickel in an amount of about 30 to about 60 wt % and niobium in an amount of about 1 to about 50 wt %, with the remainder being titanium. The weight percents are based on the total composition of the alloy used for the film. Within the range for nickel, it is generally desirable to use an amount greater than or equal to about 35, preferably greater than or equal to about 40, and more preferably greater than or equal to about 47 wt %, based on the total composition of the alloy used for the film. Also desirable within this range is an amount of nickel less than or equal to about 55, preferably less than or equal to about 50, and more preferably less than or equal to about 49 wt %, based on the total composition of the alloy used for the film. Within the above specified range for niobium, it is generally desirable to use an amount greater than or equal to about 11, preferably greater than or equal to about 12, and more preferably greater than or equal to about 13 wt %, based on the total composition of the alloy used for the film. Also desirable within this range is an amount of niobium less than or equal to about 25, preferably less than or equal to about 20, and more preferably less than or equal to about 16 wt %, based on the total composition of the alloy used for the film.

An exemplary composition of a titanium-nickel-niobium alloy is one having about 48 wt % nickel and about 14 wt % niobium, based on the total composition of the alloy used for the film. The alloy in the fully annealed state has an Aₛ temperature below the body temperature. However, when subsequently deformed with a properly controlled amount of deformation at a cryogenic temperature, the Aₛ temperature can be elevated above the ambient temperature. The cryogenic temperature as defined herein is are temperatures from about −10° C. to about −90° C. A NiTiNb alloy can therefore be fabricated in its expanded geometry, annealed and then subsequently deformed to manipulate the Aₛ temperature above the ambient.

Nickel-free shape memory alloys detailed in U.S. Pat. No. 6,258,182, the entire contents of which are incorporated by reference may also be used in the films. A preferred nickel-free β-titanium alloy generally comprises about 10 to about 12 weight percent (wt %) molybdenum, about 2.8 to about 4.0 wt % aluminum, up to about 2 wt % of chromium and vanadium, up to about 4 wt % niobium, with the balance being titanium, wherein the weight percents are based on the total weight of the composition used for the film. An exemplary nickel-free shape memory alloy is one which exhibits pseudo-elasticity between −25 and 25° C. and comprises about 10.2 wt % molybdenum, about 2.8 wt % aluminum, about 1.8 wt % vanadium, about 3.7 wt % niobium, with the balance being titanium, wherein the weight percents are based on the total weight of the composition used for the film. Another exemplary nickel-free shape memory alloy is one which exhibits pseudo-elasticity between −25 and 50° C. and comprises about 11.1 wt % molybdenum, about 2.95 wt % aluminum, about 1.9 wt % vanadium, about 4.0 wt % niobium, with the balance being titanium, wherein the weight percents are based on the total weight of the composition used for the film

Shape memory alloys, which are free of nickel, may also be used. Suitable examples of nickel free alloys are β-titanium alloys, silver-cadmium alloys, gold-cadmium alloys, copper-iron alloys, copper-aluminum-nickel, copper-tin, copper-zinc alloys such as copper-zinc-tin, copper-zinc-silicon, and copper-zinc-aluminum alloys, indium-titanium alloys, iron-platinum alloys, copper-manganese and iron-manganese-silicon alloys, and the like, as well as combinations comprising at least one of the foregoing alloys. Preferred nickel free alloys are the β-titanium alloys. The preferred shape memory alloys used for the film 200 are the Ti-55.5 wt %-Ni alloy.

The film 200 generally preferably has a thickness of about 1 to about 20 micrometers. Within this range a thickness of greater than or equal to about 2, preferably greater than or equal to about 3, and more preferably greater than or equal to about 4 micrometers may be used. Also desirable within this range is a thickness of less than or equal to about 18, preferably less than or equal to about 15, and more preferably less than or equal to about 10 micrometers. The most preferred value of film 200 thickness is 5 micrometers.

The substrate 202 generally comprises a wafer having a circular cavity 204. The cavity 204 has an average diameter proportional to the volume of insulin that is to be delivered in order to effectively maintain blood glucose levels at a desired value. The substrate 204 may be made from a wide variety of materials such as stainless steels, titanium or titanium alloys that do not have shape memory properties, glasses, silicon, or the like. Polymeric resins may also be used as a substrate. The polymeric resins may be thermoplastic resins, blends of thermoplastic resins, thermosetting resins, blends of thermosetting resins, or blends of thermoplastic with thermosetting resins. Suitable examples of thermoplastic resins that may be used in the substrate 204 include polyacetals, polyacrylics, polycarbonates, polystyrenes, polyesters, polyamides, polyamideimides, polyarylates, polyurethanes, polyarylsulfones, polyethersulfones, polyarylene sulfides, polyvinyl chlorides, polysulfones, polyetherimides, polytetrafluoroethylenes, polyetherketones, polyether etherketones, or the like, or combinations comprising at least one of the foregoing thermoplastic resins.

The preferred material for the substrate 204 is silicon because it can withstand high temperatures and can be photo-etched to produce very accurately dimensioned features, which facilitates the fabrication of the various assemblies.

The substrate 204 generally has a cavity that may be elliptical, circular, rectangular, square, rhombohedral, polygonal, or the like, in shape. The preferred shape of the cavity is circular. The total area of the cavity plays an important role in determining the volume of either insulin or the anti-inflammatory fluid that is delivered by the pump 20A or 20B. It is generally desirable for the cavity to have an area of about 5 to about 25 square millimeters (mm²). Within this range, it is desirable for the cavity to have an area of greater than or equal to about 2, preferably greater than or equal to about 4, and more preferably greater than or equal to about 5 mm². Also desirable within this range is a cavity with an area of less than or equal to about 18, preferably less than or equal to about 15, and more preferably less than or equal to about 12 mm². In the manufacturing of the pump, the film 200 is first deposited on to the substrate 202 by sputtering. The substrate 202 is then etched away to create the cavity 204. The cavity 204 is etched into the substrate by process such as physical etching, chemical etching, electron beam etching, or the like. Following the creation of the cavity 204, the exposed film is then subjected to the deformation process to create the dome shape in the film 200.

In the case of the first pump 20A, that delivers the insulin, the cavity is preferably circular in shape having a radius of about 0.2 to about 3 millimeter (mm). Within this range it is generally desirable to have a radius of greater than or equal to about 0.5, preferably greater than or equal to about 1.0 and more preferably greater than or equal to about 1.2 mm. Also desirable within this range is a radius of less than or equal to about 2.7, preferably less than or equal to about 2.5 and more preferably less than or equal to about 2.3 mm. The preferred radius of the circular cavity is 1.5 mm.

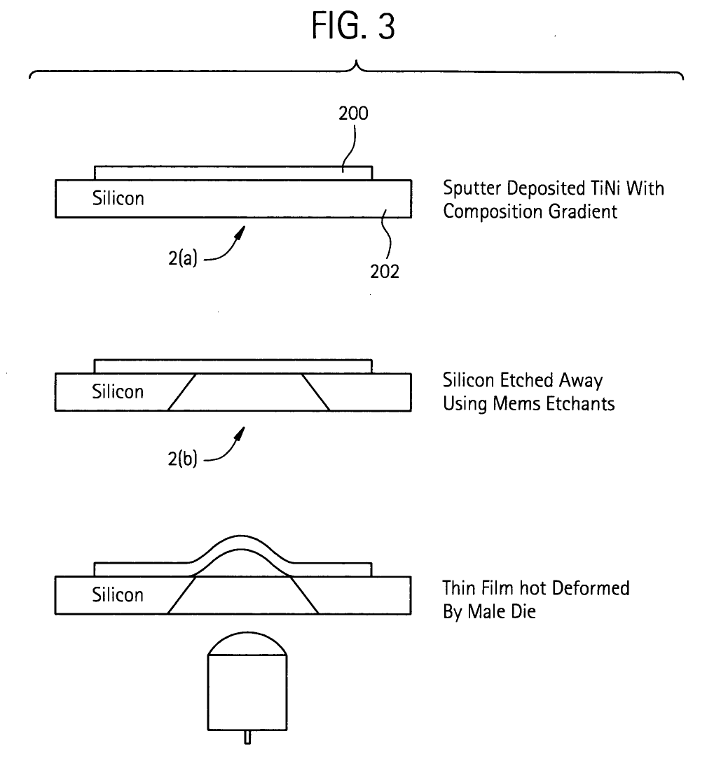

In the manufacturing of the pump 20A or 20B, the film 200 is designed to have the dome shaped contour prior to the construction of the pump 20A by deforming it into a dome shape at an elevated temperature of about 500° C. for about 30 minutes followed by subsequent cooling. The film contour is designed so that the maximum strain developed in the film is about 1%, which permits the fatigue life to be in excess of 10⁶ cycles, which corresponds to a time period of greater than or equal to about 10 years. In one exemplary embodiment, in one method of manufacturing the pumps 20A or 20B, a sputtered thin film of nickel titanium is deposited on a first surface of the silicon wafer 204. After sputtering, the surface of the wafer opposed to the first surface is etched away exposing the thin film. The exposed area of the film 200 is hot deformed at an elevated temperature of about 480° C. by a pointed probe having a spherical tip. A schematic of this operation is shown in the FIG. 3. The film 200 undergoes a martensitic transformation when cooled from the elevated temperature and this transformation imparts to the film its shape memory properties. Upon cooling the film reverts to its flat shape. The temperature difference separating the transformation temperature of the parent phase from the lower transformation temperature of the martensitic phase varies with the alloy composition and for the nickel titanium alloys that may be used in the film, it can be as much as about 30° C. When the thin film is heated by electrical current it assumes the dome shape and upon cooling this dome shape is returned to its original shape. This reciprocating motion of the film promotes the pumping action of the pump.

As stated above, the thin film is deposited on a rectangular silicon wafer and the dome is formed at the geometric center of the film. This permits adequate room for electrical contacts on the film. The film is in electrical communication with a battery that provides the electrical current for resistive heating of the film. A preferred source of electrical current is a rechargeable lithium ion battery that is charged by means of an external inductively coupled charger. Power consumption is approximately 4 milliwatts (mW) per stroke.

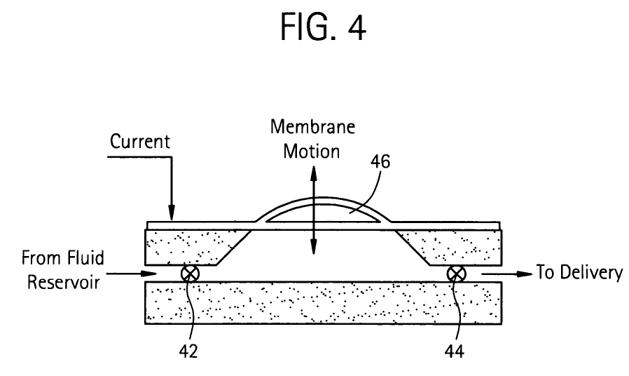

One mode of operation of a single pump 20A, 20B is illustrated schematically in FIG. 4. In the operation of the pump 20A, 20B of FIG. 4, the film 200 is heated electrically, which facilitates the formation of the dome in the film. This creates a vacuum inside the pump, which draws the fluid (either insulin or the anti-inflammatory agent) into the pump from the reservoir 32, 34 through the first check valve 42 (also termed the inlet check valve). After the fluid has entered the pump 20A, 20B, the first check valve 42 closes. The film 200 then cools facilitating the return of the film to its original shape and forcing the fluid out of the pump through the second check valve 44. The volume of insulin or the anti-inflammatory agent delivered is generally equal to the volume of the hemisphere 46 formed by the dome.

Since insulin analogs now have concentrations of about 40 to about 500 units of insulin per milliliter, a pump would have a delivery of 0.2 units per pump stroke which for a U400 insulin would require 0.5 microliters per stroke. Thus the delivery of 50 units per day would equate with 250 strokes of the pump. A stroke as defined herein is one forward and backward motion of the film 200 (i.e., the film 200 forms a dome (forward motion) once upon the application of an electrical current and returns to its flat state (backward motion) upon removal of the current). A 15 ml reservoir would have sufficient insulin for 120 days, although refilling could be carried out at shorter intervals of time that the 120 days. If the pump carries out 250 strokes per day, there would be 91,250 strokes per year. This would equate to a longevity of greater than or equal to about 1,000,000 cycles (10 years). In one exemplary embodiment related to the operation of the pump 20A, 20B, for a delivered volume of 0.5 microliters, the volume of the dome formed by the film upon the application of an electrical current would be 0.5 cubic millimeters (mm³) which equates to a cavity radius of 1.5 millimeters (mm). In this condition, the strain on the film would be about 1% indicating that a pump life expectancy in excess of 10 years may be expected.

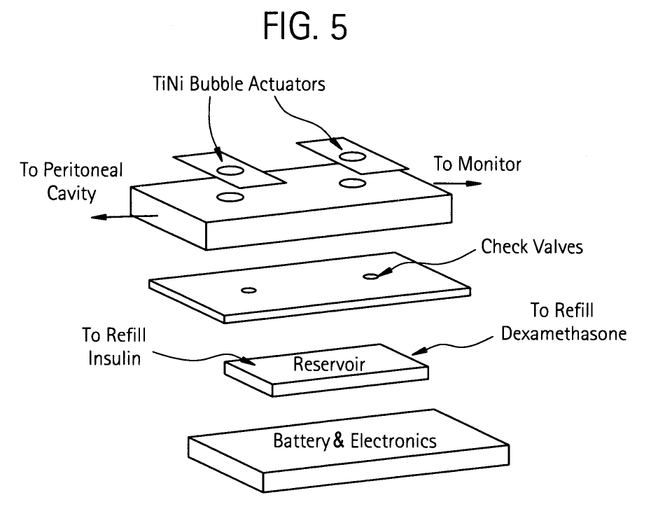

In another exemplary embodiment, a duplex pump may be used to provide a controlled delivery of insulin and an anti-inflammatory drug such as dexamethasone. The two thin film diaphragm pumps and the associated check valves, reservoir and the control circuitry and battery are assembled from four photo-lithographed silicon wafers, are shown in the FIG. 5. The duplex pump system is housed in a smoothly contoured titanium housing with ports for insulin and drug refilling and for insulin delivery to the peritoneal cavity and drug delivery to glucose monitor site. The duplex pump advantageously permits the use of a single pump device for facilitating the delivery of insulin and the anti-inflammatory agent.

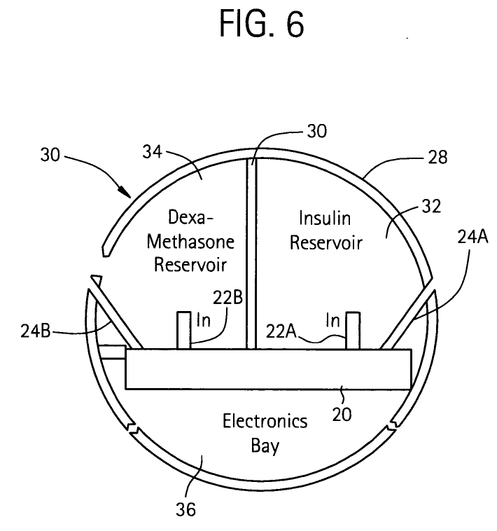

FIG. 6 shows one embodiment of an artificial pancreas employing a single duplex pump that is in fluid communication with both reservoirs. In the FIG. 6 the duplex pump 20 is in fluid communication with reservoirs 32 and 34.

Glucose monitors that use enzymatic chemistry comprise an immobilized enzyme comprising a glucose oxidase coating with an interface to an electrochemical transducer. The glucose oxidase coating on a sensor membrane catalyzes the following reaction (I)

Glucose+0 _{2→}gluconic acid+H₂0₂ (I).

The hydrogen peroxide (H₂0₂) level is directly proportional to the glucose available and is determined by a cell that measures the electrical current produced when the H₂0₂ is oxidized at the surface of a platinum (Pt) electrode. Sensors of this type have been developed using miniaturization techniques that yield a robust and relatively inexpensive sensor.

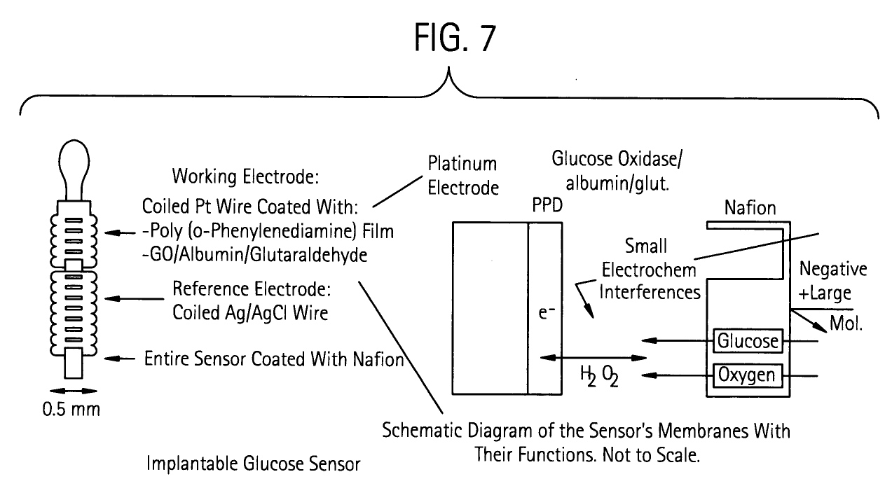

FIG. 7 represents one embodiment of a blood glucose detector for implantation within a blood vessel. The blood glucose detector comprises a glucose sensor element 40, a deviation detector or a glucose level correction control element and a microprocessor that controls the pump 20A, 20B, to deliver insulin and the anti-inflammatory agent to the peritoneal cavity of a living being. The glucose detector in conjunction with the pumps 20A, 20B and the reservoirs 32 and 34 form a closed loop. The sensor element 40 is a Nafion-containing device utilizing a two electrode design comprising a platinum (Pt) electrode 41A for monitoring the glucose and a silver/silver chloride (Ag/AgCl) reference counter electrode 41B. The three layers of the sensor are shown in FIG. 7. The sensor comprises a Nafion outer layer 42, a glucose oxidase middle layer 44 and a poly(o-phenyenediamine) (PPD) coating 46 disposed upon the Pt electrode. The PPD is permeable to the H₂0₂ generated by the oxidase reaction but impermeable to other interfering species such as ascorbic acid, uric acid, or the like. The glucose oxidase is immobilized by a bovine serum albumin/gluteraldehyde matrix. In the presence of oxygen, glucose is oxidized by the enzyme and produces hydrogen peroxide, which is oxidized at the surface of the platinum electrode, thereby producing an electric current, which is monitored. The current produced at the platinum electrode is proportional to the glucose level. This current is processed by the controller and determines the frequency of insulin delivery. The sensor has a high sensitivity to changes in the glucose levels in the blood stream and is not affected by variations in partial oxygen pressure (pO₂).

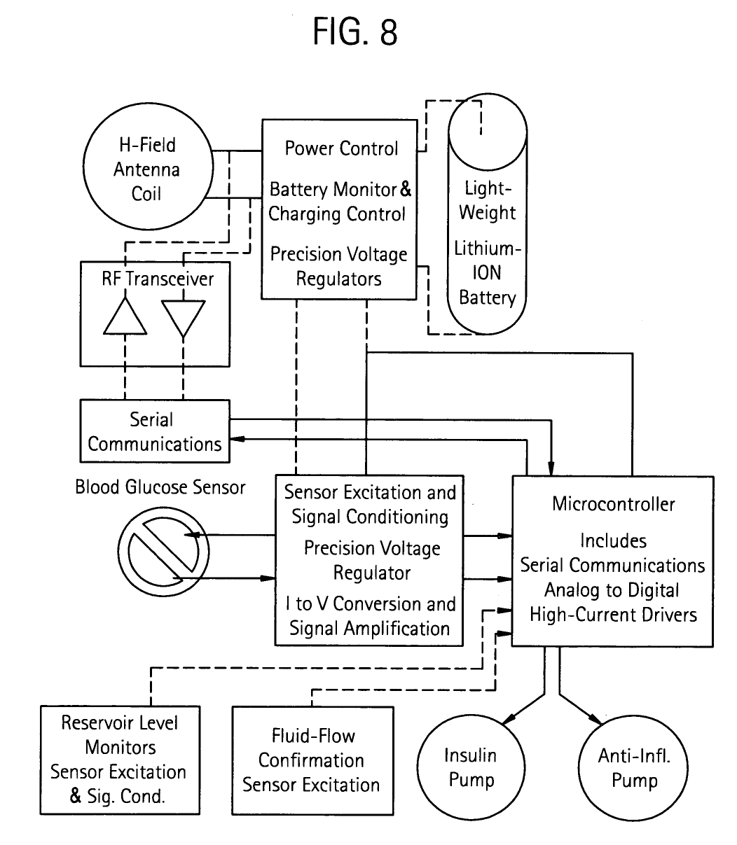

The control electronics contained in the electronics bay, provides the interface between the signal generated by the glucose monitor signal and the insulin pump, thereby creating a closed-loop system. FIG. 8 is a line diagram representing one embodiment of the control electronics that determines the amount of insulin to be delivered to the peritoneal cavity. As shown in the FIG. 8, a precision voltage source provides the excitation for the monitor, and the battery also provides the current for the heating the film 200 used in the pumps 20A, 20B. The software controls the output rate of both the insulin and the anti-inflammatory agent pumps. The insulin pump will be controlled by a standard proportional-integral-differential (PID) control algorithm. The Proportional-Integral-Differential control is used to adjust the response of the pump so that the delivery of insulin can be adjusted for a time period of about milliseconds to tens of minutes. This response of the pump is adjusted to accommodate different types of insulin that might be used, with the objective of minimizing divergence of glucose levels from the desired 5.5 mmol/l. Similarly the output of the anti-inflammatory agent pump will be selected to match a desired delivery rate in order to minimize inflammation.

The control system generally comprises a precision 0.01% temperature compensated voltage reference for sensor excitation, analog input operational amplifiers to raise the sensor voltage signal to a useful value, metal-oxide-semiconductor field effect transistor (MOSFET) switches for switching DC power to the film for resistive heating and for switching analog signals, and a sophisticated micro-controller with analog to digital circuitry, including sleep timer and electrically erasable programmable read-only memory (EEPROM).

Other functions for monitoring system performance and health may also be incorporated into the control electronics if desired, such as, telemetry of system functions to an outside monitor, a battery condition indicator, and electromagnetic coupling of the battery to an external charger.

The artificial pancreas as detailed above has a number of advantages. The high pressure capabilities of the insulin pump can be utilized to minimize clogging of the lines in the system due to insulin precipitation. If a form of insulin that displays excessive precipitation is used, the artificial pancreas advantageously permits the lines to be periodically flushed with a saline solution injected through a side arm on the capillary by transcutaneous delivery. The artificial pancreas has a long life since the simultaneous delivery of the anti-inflammatory agent to the glucose monitor prevents inflammation at the site at which the monitor is implanted. The artificial pancreas can advantageously be implanted into the body of a living being and can function without maintenance or removal from the body for extended periods of time.

The following examples, which are meant to be exemplary, not limiting, illustrate the methods of manufacturing for some of the various embodiments of the artificial pancreas described herein.

### EXAMPLES

### Example 1

In this example, the glucose monitor shown in FIG. 7 comprising a platinum electrode, a first layer of PPD disposed upon the platinum electrode, a second layer of glucose oxidase in a matrix of bovine serum albumin (BSA) and glutaraldehyde disposed upon the layer of PPD, and a third layer of NAFION® disposed upon a surface of the second layer opposed to the surface in contact with the layer of PPD was implanted into dogs to determine the in vivo effect on the life cycle of the sensor. While the sensor showed a linear response, high sensitivity to blood glucose levels and a fast response time, it deteriorated rapidly.

Experiments were then conducted with a glucose monitor conditioned at temperature of 120° C. The glucose oxidase (GO), when immobilized in a matrix of BSA and glutaraldehyde, can withstand a temperature of 120° C. without a loss of activity, and is thus compatible with the conditioning procedure established for Nafion. The thermally annealed glucose monitor showed a linear response up to at least 20 millimoles (mM) glucose, and a slope of 3.2 nanoamperes/millimole (nA/mM) with an intercept of 5.7 nA. The response time of the monitor was about 30 seconds and the time required for the background current to decay to steady state after initial polarization was about 35 min. The sensor had a high selectivity for glucose and low pO₂ levels affected the response of the sensor only for levels below 8 mm mercury (Hg).

The thermally annealed monitors were evaluated in vivo by implanting in the backs of dogs and testing regularly over a 10 day period. About 45 minutes after polarization in vivo, the current started to stabilize. After this period, a bolus intravenous injection of glucose was made and the sensor output was monitored. Blood was periodically sampled from an indwelling catheter to determine blood glucose levels. A 5 to 10 minute delay between the maxima in blood glucose and the sensor's signal was observed, corresponding to the known lag time between blood and subcutaneous glucose levels. Although experiments with dogs showed that the response of some monitors remained stable for at least 10 days, others failed due to the tissue reactions. Thus, it was determined that controlling the composition of the monitor as well as the tissue microenvironment could prolong the lifetime of the monitors in vivo.

In the efforts to characterize baseline tissue reactions, the current unmodified Nafion-containing glucose monitor was implanted in Sprague-Dawley rats and tissue samples were obtained one day and one month post implantation. The specimens were processed for traditional histopathology using Hematoxylin and Eosin (H & E) Staining, as well as trichrome staining (fibrin and collagen deposition). At one-day post-implantation, a massive inflammatory reaction was induced at the tissue site surrounding the sensor, comprised primarily of polymorphonuclear (PMN) and mononuclear leukocytes, as well as fibrin deposition. By one-month post-implantation, significant chronic inflammation and fibrosis was present around the sensor, and the presence of mature collagen and activated fibroblasts with associated loss of vasculature was also noted. It was felt that alteration of the tissue microenvironments surrounding the sensor via locally administered Tissue Response Modifiers (e.g. anti-inflammatory drug) would likely have a major positive effect on the architecture of the tissue (i.e. decreased inflammation and fibrosis) that may extend the glucose sensor lifetime.

### Example 2

This example was undertaken to minimize, as well as to try to stop, the inflammatory and fibrotic reactions to an implant in rats using dexamethasone-polylactic co glycolic acid microspheres (PLGA microspheres) for continuous delivery of dexamethasone. Using a mixed system of un-degraded and pre-degraded microsphere formulations as well as free drugs, a continuous release profile of the drug was obtained. This microsphere system was then tested in vivo and in vitro in rats.

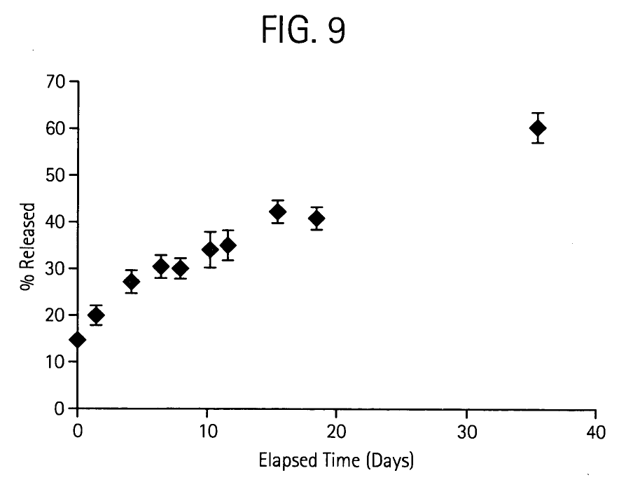

In Vitro Dexamethasone Release from PLGA microspheres: The focus of this research study was to develop polylactic-co-glycolic acid (PLGA) microspheres for continuous delivery of dexamethasone for over a one month period, in an effort to suppress the acute and chronic inflammatory reactions to implants such as biosensors, which interfere with their functionality. The microspheres were prepared using an oil/water emulsion technique. The oil phase was composed of 9:1 dichloromethane to methanol with dissolved PLGA and dexamethasone. Some dexamethasone PLGA microspheres were pre-degraded for one or two weeks. The in vitro release studies were performed at a constant temperature (37° C.), in phosphate buffered saline at sink conditions. Drug loading and release rates were determined by high performance liquid chromatography-ultraviolet (HPLC-UV) analysis. The standard (un-degraded) microsphere systems did not provide the desired release profile since, following an initial burst release, a delay of two weeks occurred prior to continuous drug release. Predegraded microspheres started to release dexamethasone immediately but the rate of release decreased after only 2 weeks. Thus, a mixture of standard and pre-degraded microspheres was used to avoid this delay and to provide continuous release of dexamethasone for one month, as shown in FIG. 9.

In Vivo Dexamethasone Release from PLGA Microspheres: The purpose of this research study was to evaluate in vivo the newly developed dexamethasone/PLGA microsphere system described above that was designed to suppress the inflammatory and fibrotic responses to an implanted device such as a glucose monitor. The microspheres were prepared as described above and were composed of drug-loaded microspheres (including newly formulated and pre-degraded microspheres) as well as free dexamethasone. The efficacy of the mixed microsphere system to control the tissue reactions to an implant were then tested in vivo using cotton thread sutures as a model. Sutures were chosen as model sensors in lieu of the glucose monitor of the aforementioned experiments since histology is much easier to perform with cotton threads than sensors due to the difficulty of sectioning through the metal components of the sensor. Sutures of cotton thread were used in vivo to induce inflammation subcutaneously in Sprague-Dawley rats. Two different in vivo studies were performed; the first was to determine the effective dosage level of dexamethasone to suppress the acute inflammatory reaction and the second was to show the effectiveness of the dexamethasone delivered by PLGA microspheres to suppress the chronic inflammatory response to an implant.

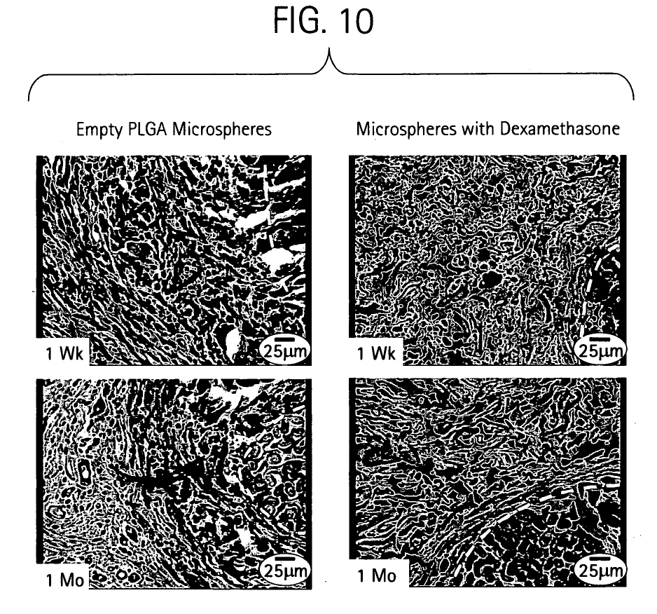

The first in vivo study showed that 0.1 to 0.8 mg of dexamethasone at the site of implantation minimized the acute inflammatory reaction. The second in vivo study demonstrated that our mixed microsphere system suppressed the inflammatory response to an implanted suture for at least one month as shown in FIG. 10. This study has proven the viability of microsphere delivery of an anti-inflammatory drug to control the inflammatory reaction at an implant site. Evaluation of efficacy of the dexamethasone/PLGA microsphere system in suppressing inflammation to a thread suture was used as model sensor. As seen in the FIG. 10, the photomicrographs on the left are sites with thread and empty PLGA microspheres that have been implanted for one week and one month respectively. The photomicrographs on the right are sites with thread and dexamethasone loaded PLGA microspheres that have been implanted for one week and one month respectively. The inflammatory response to the thread suture has been significantly suppressed by the dexamethasone loaded microspheres.

However, it was determined that the use of the PLGA system to deliver dexamethasone is not entirely functional for two reasons. The first reason is that since PLGA degrades to acidic products, the microspheres themselves induce inflammation caused by the low pH. The second reason is the low incorporation of dexamethasone in the microspheres, which consequently results in having to implant a large volume of microspheres. This large volume of implanted microspheres also promotes inflammation.

The above experiments show that the artificial pancreas comprising a pump for delivering insulin as well as an anti-inflammatory agent and a glucose monitor is a closed cycle system that can be utilized in living beings for extended periods of time. The use of the pump to deliver the anti-inflammatory agent minimizes the growth of tissue, which reduces the life cycle of the glucose monitor. Additionally the pump can deliver insulin on demand and therefore reduce hyperglycemia or hypoglycemia as well as other advanced disorders that result from blood glucose levels not being continuously maintained at desired values.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention.

What is claimed is:

1. An artificial pancreas comprising:
a first reservoir for retaining insulin;
at least one second reservoir for retaining a therapeutic agent;
at least one pump in fluid communication with the first reservoir and the at least one second reservoir; and
a glucose monitor in electrical communication with the pump.

2. The artificial pancreas of claim 1, wherein the artificial pancreas in enclosed in a biocompatible housing.

3. The artificial pancreas of claim 2, wherein the housing comprises titanium or a titanium alloy.

4. The artificial pancreas of claim 2, wherein the housing comprises a polymeric resin.

5. The artificial pancreas of claim 4, wherein the housing comprises polyamides, polytetrafluoroethylene, silicone polymers, polyolefins, nonabsorbable polyesters, homopolymers and copolymers of lactic acid, glycolic acid, lactide, glycolide, para-dioxanone, trimethylene carbonate, ∈-caprolactone, or biocompatible combinations comprising at least one of the foregoing polymeric resins.

6. The artificial pancreas of claim 1, wherein the first reservoir and the at least one second reservoir are not in fluid communication with one another.

7. The artificial pancreas of claim 1, wherein the first reservoir has a volume of 5 to about 50 milliliters.

8. The artificial pancreas of claim 1, wherein the first and second reservoirs are refilled using a transcutaneous hypodermic injection.

9. The artificial pancreas of claim 1, wherein the pump is in fluid communication with the first reservoir and the second reservoir via a check valve.

10. The artificial pancreas of claim 1, wherein the pump can deliver fluid at pressures of greater than or equal to about 25 pounds per square inch.

11. The artificial pancreas of claim 1, wherein the pump can deliver fluid at pressures of greater than or equal to about 75 pounds per square inch.

12. The artificial pancreas of claim 1, wherein the at least one pump is a duplex pump.

13. The artificial pancreas of claim 1, wherein the at least one pump is a triplex pump.

14. The artificial pancreas of claim 1, wherein the pump comprises at least one film of shape memory alloy disposed upon a substrate, and further wherein the substrate has at least one cavity.

15. The artificial pancreas of claim 14, wherein the film is a nickel titanium alloy.

16. The artificial pancreas of claim 15, wherein the nickel titanium alloy comprises about 54.5 to about 57 wt % nickel, based on the total composition of the alloy.

17. The artificial pancreas of claim 14, wherein the film is in electrical communication with a source of electrical power.

18. The artificial pancreas of claim 17, wherein the source of electrical power is a rechargeable lithium ion battery that is charged by means of an external inductively coupled charger and further the power consumption by the film is approximately 4 milliwatts per stroke.

19. The artificial pancreas of claim 14, wherein the film operates at a maximum strain of about 1%, and wherein the fatigue life of the film is in excess of 10⁶ cycles.

20. The artificial pancreas of claim 14, wherein the substrate is a polymeric resin derived from a thermoplastic resin, a blend of thermoplastic resins, a thermosetting resin, a blend of thermosetting resins, or blends of thermoplastic with thermosetting resins.

21. The artificial pancreas of claim 14, wherein the substrate has at least one circular cavity having a radius of 1 to 20 millimeter.

22. The artificial pancreas of claim 14, wherein the substrate has two cavities.

23. The artificial pancreas of claim 14, wherein the substrate comprises silicon and has a cavity that is elliptical, circular, rectangular, square, rhombohedral, or polygonal in shape.

24. The artificial pancreas of claim 14, wherein the substrate comprises a cavity and wherein the cavity is about 2 to about 20 square millimeters in area.

25. The artificial pancreas of claim 14, wherein the volume of a dome formed by the film upon the application of an electrical current is 0.5 cubic millimeters.

26. The artificial pancreas of claim 1, wherein the pump is in fluid communication with at least two reservoirs.

27. The artificial pancreas of claim 1, comprising at least two pumps.

28. The artificial pancreas of claim 1, wherein the therapeutic agent is an anti-inflammatory agent, and wherein the anti-inflammatory agent is dexamethasone, prednisolone, corticosterone, budesonide, estrogen, sulfasalazine, and mesalamine.

29. The artificial pancreas of claim 1, wherein the glucose monitor is in electrical communication the pump via an electrical control system.

30. The artificial pancreas of claim 1, wherein the glucose monitor comprises a two electrode design comprising a platinum electrode for monitoring the glucose and a silver/silver chloride reference counter electrode.

31. The artificial pancreas of claim 1, wherein the glucose monitor comprises a sensor comprising a cation exchange polymer outer layer, a glucose oxidase layer and a poly(o-phenyenediamine) (PPD) coating disposed upon a platinum electrode.

32. The artificial pancreas of claim 31, wherein the cation exchange polymer is a sulfonated polytetrafluoroethylene.

33. The artificial pancreas of claim 1, comprising a close loop insulin deliver system.
